(19) United States
(12) Patent Application Publication
(10) Pub. No.: US 2024/0389216 A1
(43) Pub. Date: Nov. 21, 2024

(54) PLASMA-BASED AIR DISINFECTION AND FILTRATION SYSTEM

(71) Applicants: Raj Kamal Singh, Columbia, PA (US); Hema Priyamvada Ravindran, Columbia, PA (US)

(72) Inventors: Raj Kamal Singh, Columbia, PA (US); Hema Priyamvada Ravindran, Columbia, PA (US)

(21) Appl. No.: 18/788,254

(22) Filed: Jul. 30, 2024

(51) Int. Cl. H05H 1/34; A61L 9/22; B03C 3/019; B03C 3/36; B03C 3/38; B03C 3/41; B03C 3/49; B03C 3/88

(52) U.S. Cl. CPC H05H 1/3478; A61L 9/22; B03C 3/019; B03C 3/361; B03C 3/38; B03C 3/41; B03C 3/49; B03C 3/88; A61L 2209/14; H05H 2245/10

(57) ABSTRACT

The disclosure provides an air disinfection and filtration apparatus for the removal of particles and disinfection of a flow of air using plasma. The apparatus comprises an electrode section with coaxial electrodes that form an annular cavity between the coaxial electrodes. Further, the apparatus comprises an ionic thruster assembly connected to the annular cavity of the electrode section. The ionic thruster assembly includes a top inlet and is configured to receive an airflow, charge the particles within the airflow, and transmit the airflow with the charged particles into the annular cavity of the electrode section. Further, the electrode section is configured to attract the charged particles towards an outer electrode of the coaxial electrodes. The apparatus further comprises a swirl generation unit, a catalytic bed, a particle collector, a collector adaptor, and a dome-shaped closure member.

### TECHNICAL FIELD

The present disclosure relates to a system, method, and apparatus for disinfection and filtration of air using plasma.

### BACKGROUND

Maintaining high air quality and controlling airborne pathogens are critical concerns in various environments, such as healthcare facilities, industrial settings, and residential areas. Traditional air filtration systems, while capable of removing particulate matter, often fall short in effectively neutralizing airborne pathogens and other contaminants. This shortcoming has become particularly pronounced with the increasing prevalence of infectious diseases and rising pollution levels.

Existing air purification technologies primarily rely on mechanical filtration methods, such as High-Efficiency Particulate Air (HEPA) filters, MERV (Minimum Efficiency Reporting Value) filters, and activated carbon filters. While these systems are effective in capturing particulate matter, they present several drawbacks. Firstly, mechanical filters can trap pathogens but do not neutralize them, which means there is a risk of recontamination during filter replacement or maintenance. Secondly, these filters can become breeding grounds for microbes, leading to microbial colonization on the filter surfaces. This microbial growth can act as a potential source of bioaerosol spore emission and transmission, potentially compromising indoor air quality. Additionally, these traditional filters struggle to eliminate volatile organic compounds (VOCs) and other gaseous pollutants, which require additional treatment methods for effective removal.

Furthermore, mechanical filters require regular replacement or cleaning to maintain efficiency, leading to increased operational costs and downtime. The resistance to airflow created by HEPA and similar filters necessitates the use of powerful fans, which increases energy consumption to maintain adequate air circulation. Moreover, the effectiveness of mechanical filters diminishes over time as they become clogged with particles, reducing their filtration capacity and necessitating frequent replacements.

Another issue with some air purification systems, such as ultraviolet (UV) germicidal irradiation, is the production of ozone and ultrafine particles when used indoors as byproducts. These byproducts can be harmful to human health and requires additional measures to mitigate its presence. Direct exposure to UV-C light can cause severe eye injuries, including photokeratitis (corneal inflammation), and skin damage, such as erythema (sunburn). Moreover, Prolonged or repeated exposure to UV-C radiation can increase the risk of skin cancer. These limitations highlight the need for advanced air purification technologies that can provide comprehensive disinfection, efficient pollutant removal, and low maintenance requirements.

Continuous Positive Airway Pressure (CPAP) machines, used for treating sleep apnea, also face significant challenges regarding air filtration. The filters commonly used in CPAP machines are often not efficient enough to remove submicron bioaerosols, which are a respiratory concern. This inefficiency is largely due to the significant pressure drop that occurs when using high-efficiency filters such as MERV 14+ or HEPA filters. Consequently, these machines may fail to adequately protect users from inhaling harmful bioaerosols, potentially exacerbating respiratory conditions and undermining the therapeutic benefits of the device.

Therefore, there is a need for a system and apparatus that may overcome the drawbacks of current technologies.

### SUMMARY

This summary is provided to introduce a selection of concepts in a simplified form that are further disclosed in the detailed description of the invention. This summary is not intended to identify key or essential inventive concepts of the claimed subject matter, nor is it intended to determine the scope of the claimed subject matter.

The present disclosure discloses an apparatus for air disinfection and filtration using plasma to overcome the above-stated limitations of the existing technologies.

An aspect of the present disclosure relates to a plasma-based fluid disinfection and filtration apparatus. The apparatus, according to the present aspect, is provided with an electrode section comprising coaxial electrodes with an annular cavity between them. The apparatus also includes an ionic thruster assembly that may be in fluid communication with the annular cavity of the electrode section at one end.

Further, the ionic thruster assembly may comprise a top inlet that is designed to receive airflow through the inlet and charge the particles present within the incoming airflow. Further, the airflow with the charged particles may be transmitted into the annular cavity of the electrode section. The electrode section is further configured to attract the charged particles towards an outer electrode of the coaxial electrodes.

Further, the coaxial electrodes may include an outer electrode and an inner electrode wherein both the outer electrode and the inner electrode may be hollow in shape. Further, the diameter of the outer electrode may be larger than the diameter of the inner electrode to define the annular cavity between the electrodes. In one embodiment, the coaxial electrodes may be cylindrical in shape. In another embodiment, the coaxial electrode may be conical in shape.

The ionic thruster assembly may include an inner cylindrical wall, a coaxial outer cylindrical wall, and an annular space defined between the inner cylindrical wall and the outer cylindrical wall. The ionic thruster assembly may further include a plurality of electrodes between the inner cylindrical wall and the outer cylindrical wall. Further, the plurality of electrodes may be configured throughout the periphery of the inner cylindrical wall within the annular space wherein the plurality of electrodes may include at least one charged electrode and at least one ground electrode.

Further, the ionic thruster assembly is configured to generate plasma within the annular space between the inner cylindrical wall and the outer cylindrical wall when a potential difference is applied between the charged electrodes and the ground electrodes.

In another embodiment, the ionic thruster assembly may include a duct with an internal cavity, an opening at a top end, and an opening at a bottom end opposite to the top end. Further, the internal cavity may comprise a plurality of electrodes. Further, the duct can be square, circular, or rectangular in shape. Further, the plurality of electrodes may include at least one charged electrode and at least one ground electrode.

According to the present aspect, the apparatus further includes a swirl generation unit configured between the electrode section and the ionic thruster assembly. The swirl generation unit may include an annular-shaped main body and a plurality of static vanes where the vanes may be equidistantly configured to cover the annular space of the annular-shaped main body.

The swirl generation unit may be configured to generate a swirl motion of the airflow with the charged particles within the annular cavity between the coaxial electrodes. Further, the swirl generation unit may direct the airflow toward the outer electrode of the coaxial electrodes.

Further, the inner electrode and the outer electrode may be applied with a large potential difference of opposite polarities which may attract the charged particles present within the airflow to an inner surface of the outer electrode.

The inner electrode may further include a plurality of apertures within the periphery and an outlet at an end opposite to the first end of the electrode section. Further, the apertures may be configured near the outlet of the inner electrode. Further, the apertures are configured to transmit the airflow from the annular cavity between the coaxial electrodes to an inner cavity of the inner electrode. Further, an outlet may be configured to exhaust the airflow out of the apparatus.

In an embodiment, the apparatus may further include a catalytic bed configured in the outlet of the inner electrode. The catalytic bed is configured to eliminate ozone from the exhausted airflow.

In an embodiment, the apparatus may further include a particle collector that may be configured at an opposite end to the first end of the electrode section. The particle collector may be configured to collect the charged particles attracted to the outer electrode. Further, the particle collector may be in an annular in shape and configured to detachably connect to the annular cavity of the electrode section through a magnetic coupling.

Further, the particle collector may comprise two mirrored semi-circular halves. The two halves are detachably connected to form the annular-shaped particle collector.

Further, a collector adaptor may be configured between the particle collector and the electrode section to break a swirl motion within the annular cavity of the electrode section.

In one embodiment, the apparatus further includes a dome-shaped closure member that may be configured to enclose the top opening of the inner electrode. The dome-shaped closure member may be configured to guide airflow into the annular cavity between the coaxial electrodes.

### BRIEF DESCRIPTION OF DRAWING

FIG. 1 is a cut-sectional view of an apparatus for air disinfection and filtration, according to an embodiment of the present disclosure.

FIG. 2 shows a top view of an electrode section of the apparatus, according to an embodiment of the present disclosure.

FIG. 3A shows a top view of an ionic thruster assembly of the apparatus, according to an embodiment of the present disclosure.

FIG. 3B is a cut-sectional view of the ionic thruster assembly of the apparatus, according to an embodiment of the present disclosure.

FIG. 4 is a perspective view of the ionic thruster assembly of the apparatus, according to another embodiment of the present disclosure.

FIG. 5A is a perspective view of an electrode configuration for a rectangular-shaped ionic thruster assembly of the ionic thruster assembly, according to another embodiment of the present disclosure.

FIG. 5B is a top view of the ionic thruster assembly as shown in FIG. 5A, according to another embodiment of the present disclosure.

FIG. 6A is a top view of a swirl generation unit of the apparatus, according to an embodiment of the present disclosure.

FIG. 6B is a cross-sectional view of the swirl generation unit of FIG. 6A, according to an embodiment of the present disclosure.

FIG. 7 is a perspective view of a semi-circular half of a particle collector, according to an embodiment of the present disclosure.

FIG. 8A is a perspective view of a catalytic bed and/or absorbent bed of the apparatus, according to an embodiment of the present disclosure.

FIG. 8B is a cross-sectional view of the catalytic bed and/or absorbent bed as shown in FIG. 8A, according to an embodiment of the present disclosure.

FIG. 9A is a top view of a collector adaptor of the apparatus, according to an embodiment of the present disclosure.

FIG. 9B is a side cross-sectional view of the collector adaptor of FIG. 9A, according to an embodiment of the present disclosure.

FIG. 10 is a cut-sectional view of an apparatus for air disinfection and filtration, according to another embodiment of the present disclosure.

FIG. 11A is a perspective view of a connecting member of the apparatus, according to another embodiment of the present disclosure.

FIG. 11B is a front view of the connecting member as shown in FIG. 11A, according to another embodiment of the present disclosure.

### DETAILED DESCRIPTION

In the following description, numerous specific details are set forth in order to provide a thorough understanding of the embodiments of the present invention. It will be apparent to one skilled in the art that embodiments of the present invention may be practiced without some of these specific details.

Embodiments of the present invention include various steps, which will be described below. The steps may be performed by hardware components or may be embodied in machine-executable instructions, which may be used to cause a general-purpose or special-purpose processor programmed with the instructions to perform the steps. Alternatively, steps may be performed by a combination of hardware and or by human operations.

If the specification states a component or feature “may”, “can”, “could”, or “might” be included or have a characteristic, that particular component or feature is not required to be included or have the characteristic.

As used in the description herein and throughout the claims that follow, the meaning of “a”, “an”, and “the” includes plural reference unless the context clearly dictates otherwise. Also, as used in the description herein, the meaning of “in” includes

“on” unless the context clearly dictates otherwise.

Exemplary embodiments will now be described more fully hereinafter with reference to the accompanying drawings, in which exemplary embodiments are shown. This invention may, however, be embodied in many different forms and should not be construed as limited to the embodiments set forth herein. These embodiments are provided so that this invention will be thorough and complete and willfully convey the scope of the invention to that ordinary skill in the art. Moreover, all the statements herein reciting embodiments of the invention, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure).

While embodiments of the present invention have been illustrated and described, it will be clear that the invention is not limited to these embodiments only. Numerous modifications, changes, variations, substitutions, and equivalents will be apparent to those skilled in the art, without departing from the spirit and scope of the invention, as described in the claim.

The disclosure discloses a system and an apparatus to disinfect and filter air using a non-thermal plasma and swirl separation. The inlet fluid may be polluted or infected air, gases, or a mixture of the same.

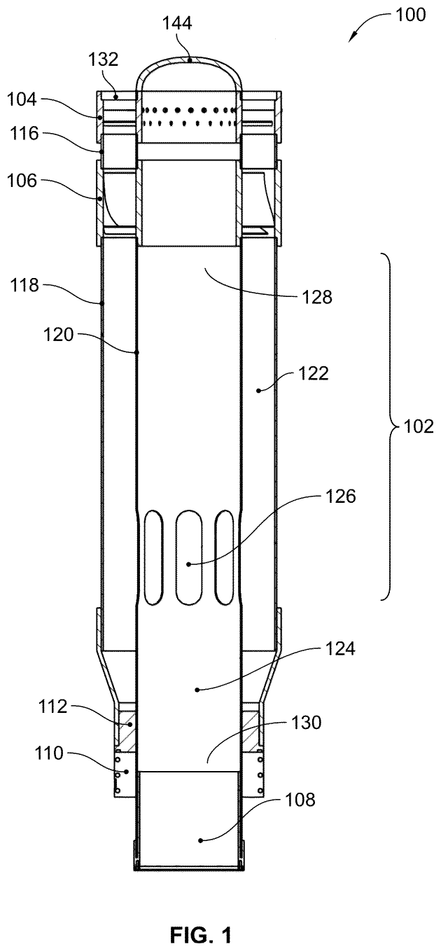

FIG. 1 is a cross-sectional view of an apparatus 100 for air disinfection and filtration, according to an embodiment of the present disclosure.

Referring to FIG. 1, the apparatus 100 comprises an electrode section 102 and an ionic thruster assembly 104 connected in a fluid communication with the electrode section 102. The ionic thruster assembly 104 is configured to receive and disinfect the inlet airflow using non-thermal plasma. Further, the ionic thruster assembly 104 may be configured to charge particles present within the airflow. The ionic thruster assembly 104 may include an inlet 132 for airflow. Further, the ionic thruster assembly 104 may include a plasma generation region and a plurality of electrodes within the plasma generation region. The plurality of electrodes includes charged electrodes and ground electrodes to generate a non-thermal plasma within the plasma generation region. The generated plasma in the plasma generation region is used to disinfect the inlet airflow and charge the particles present within the airflow. The electrode section 104 may be configured to attract the charged particles from the airflow and discharge the filtered airflow without the particles.

The electrode section 102 includes a pair of elongated cylindrical electrodes including an outer electrode 118 and an inner electrode 120. The outer electrode 118 and the inner electrode 120 may be coaxial hollow electrodes with different diameters to define an annular cavity 122 in between. For example, the diameter of the outer electrode 118 is larger than the diameter of the inner electrode 120. The elongated electrodes enable the airflow to spend more time within the annulus cavity 122 before exhausting out of the apparatus 100.

The outer electrode 118 includes a hollow cylindrical body with an opening at each end. Further, the inner electrode 120 includes a hollow cylindrical body coaxial to the outer electrode 118. The coaxial arrangement of the outer electrode 118 and the inner electrode defines an annular cavity 122 between the outer electrode 118 and the inner electrode 120 and an internal cavity 124 within the inner electrode 120. Further, the inner electrode 120 includes a first opening at a first end 128 of the electrode section 102, and a second opening at a second end 130 opposite end to the first end 128 of the electrode section 102. Further, the hollow inner electrode 120 includes a plurality of apertures 126 near the second end 130. The plurality of apertures 126 may be spread across a periphery of the inner electrode 120.

In another embodiment, the outer electrode 118 and the inner electrode 120 may be coaxial conical electrodes.

The apparatus 100 further comprises a swirl generation unit 106 mounted on the electrode section 102. The swirl generation unit 106 may be configured between the electrode section 102 and the ionic thruster assembly 104. The swirl generation unit 106 may be mounted on the electrode section 102 from one end and coupled to the ionic thruster assembly 104 from an opposite end. In one embodiment, the apparatus 100 may include a connecting adapter 116 to fluid communicatively connect the swirl generation unit 106 with the ionic thruster assembly 104. In another embodiment, the swirl generation unit 106 and the ionic thruster assembly 104 may be directly connected without the connecting adapter 116. In one embodiment, the ionic thruster assembly 104 and the swirl generation unit 106 may be made as a single unit.

The swirl generation unit 106 may be configured to generate a swirl motion of the airflow within the annular cavity 122 between the outer electrode 118 and the inner electrode 120. Further, the swirl generation unit 106 may direct the airflow with the charged particles from the ionic thruster assembly 104 towards the outer electrode 118 within the annular cavity 122 so that the charged particles may get attracted and stick to an inner surface of the outer electrode 118.

In present embodiment, the ionic thruster assembly 104 and the swirl generation unit 106 may be made in an annular shape. Further, the size of the ionic thruster assembly 104 and the swirl generation unit 106 may be the same as a size of the annular space 122 such that an annulus space between the ionic thruster assembly 104 and the swirl generation unit 106 may align with the annular cavity 122 when configured together. In one embodiment, the ionic thruster assembly 104 and the swirl generation unit 106 may be detachably mounted on the electrode section 102. In another embodiment, the electrode section 102, the ionic thruster assembly 104, and the swirl generation unit 106 are made as a single unit. The positions of ionic thruster assembly 104 and swirl generation unit can be swapped.

In another embodiment, the ionic thruster assembly 104 and the swirl generation unit 106 may be made in any other geometrical shape including, but not limited to, cylindrical, conical, square, circular, and rectangular. The ionic thruster assembly 104 and the swirl generation unit 106 are configured such that the inner cavity of the ionic thruster assembly 104 and the swirl generation unit 106 are in fluid communication with the annular cavity 122 when configured with the electrode section 102.

The ionic thruster assembly 104 comprises an opening at an end opposite to the end where the swirl generation unit 106 is connected via the connecting adapter 116. The opening of the ionic thruster assembly 104 is configured as the inlet 132 to receive the airflow within the ionic thruster assembly 104. The ionic thruster assembly 104 may disinfect the air and charge the particles present within the air using the non-thermal plasma. The non-thermal plasma may be generated through an application of a large potential difference between the electrodes of the ionic thruster assembly 104.

Since the ionic thruster assembly 104 is connected in fluid communication with an annular cavity 122 of the electrode section 102 via the swirl generation unit 106, the swirl generation unit 106 generates a swirl motion of the airflow within the annular cavity. Further, a potential difference is applied between the outer electrode 118 and the inner electrode 120. The potential of the outer electrode 118 and the inner electrode 120 is selected such that the charged particles (charged by the ionic thruster assembly 104) present within the airflow may attract towards the outer electrode 118.

Further, the airflow within the annular space 122 transmits from the first end 128 towards the second end 130 opposite to the first end 128 in the swirl motion.

Further, the plurality of apertures 126 are configured across the periphery near the second end of the inner electrode 120. The plurality of apertures 126 within the inner electrode 120 are configured to transmit the disinfected and filtered air present within the annular cavity 122 into the inner cavity 124 of the inner electrode 120.

The disinfected and filtered air exhaust out of the inner cavity 124 of the inner electrode 120 through the second opening at the second end 130. The second opening is configured as an outlet for the disinfected and filtered airflow to exhaust out of the apparatus 100.

The apparatus 100 further comprises a catalytic/adsorbent bed 108 connected to the inner electrode 120. The catalytic/adsorbent bed 108 is configured at the outlet of the inner electrode 120. Further, the catalytic/adsorbent bed 108 is configured to eliminate ozone from the exhaust airflow. The catalytic/adsorbent bed may include an absorbent, an ozone decomposition catalyst, or a combination to absorb the ozone from the exhaust airflow.

The apparatus 100 further comprises a particle collector 110 and a collector adaptor 112. The particle collector 110 and the collector adaptor 112 are connected at the second end 130 of the electrode section 102 covering a bottom opening of the annular cavity 122. The particle collector 110 may be connected to the electrode section 102 through the collector adaptor 112. The collector adaptor 112 may be configured to break the swirl motion of the airflow within the annular cavity 122 at the second end 130. Further, the particle collector 110 may be configured to detachably seal the second end 130 of the annular cavity 122. The particle collector 110 may be configured to collect the particles separated from the airflow and attracted towards the outer electrode 118. Further, the particle collector 110 may be detached to clean the collected particles.

The apparatus 100 further comprises a dome-shaped cap 114 mounted on the ionic thruster assembly 104. The dome-shaped cap 114 is configured to cover the inner cavity of the ionic thruster assembly 104 and the internal cavity 124 of the electrode section 102. Further, the dome-shaped cap 104 is designed aerodynamically to guide the air inside the annular cavity 122 without causing significant turbulence and resistance.

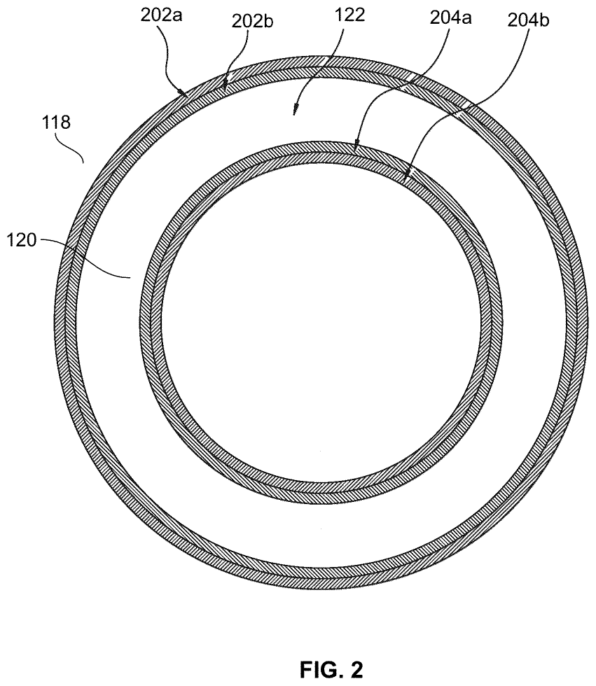

FIG. 2 shows a top view of an electrode section 102 of the apparatus 100, according to an embodiment of the present disclosure.

Referring to FIG. 2, the electrode section 102 comprises coaxial electrodes having an outer electrode 118, an inner electrode 120, and an annular cavity 122 between the outer electrode 118 and the inner electrode 120. The coaxial electrodes may be cylindrical electrodes or conical electrodes. Each electrode of the coaxial electrodes may further comprise a conductive layer and a dielectric layer. The conductive layer may be affixed to the dielectric layer to form an electrode. In the present embodiment, the outer electrode 118 includes an outer layer 202a of a dielectric material and an inner layer 202b of a conductive material. The inner layer 202b is made of a conductive material to attract the charged particles from the airflow when applied with a potential. Further, the inner electrode 120 includes an outer layer 204a and an inner layer 204b. The outer layer 204a is a conductive layer made of a conductive material and the inner layer 204b is a dielectric layer made of a dielectric material.

Therefore, the electrode section 102 includes conductive layers of the outer electrode 118 and the inner electrode 120 within the annular cavity 122.

The inner layer 202b of the outer electrode 118 and the outer layer 204a of the inner electrode 120 may be connected to a power source (not shown) that may generate a potential difference between the outer electrode 118 and the inner electrode 120. The polarity of the potential applied to the inner layer 202b of the outer electrode 118 may be opposite to the polarity applied to the outer layer 204a of the inner electrode 120. Further, the polarity may be selected such that the charged particles present within the airflow may attract to the inner layer 202b of the outer electrode 118. The conductive material may include copper, silver, aluminum, lead, platinum, or their alloy. The dielectric material may include glass, quartz, ceramics, or polymers.

In another embodiment, the coaxial electrodes (118 & 120) may be made of a conductive material.

The outer electrode 118 and the inner electrode 120 may also be configured to generate plasma within the annular cavity 122. The plasma within the annular cavity 122 may be used to further disinfect the airflow.

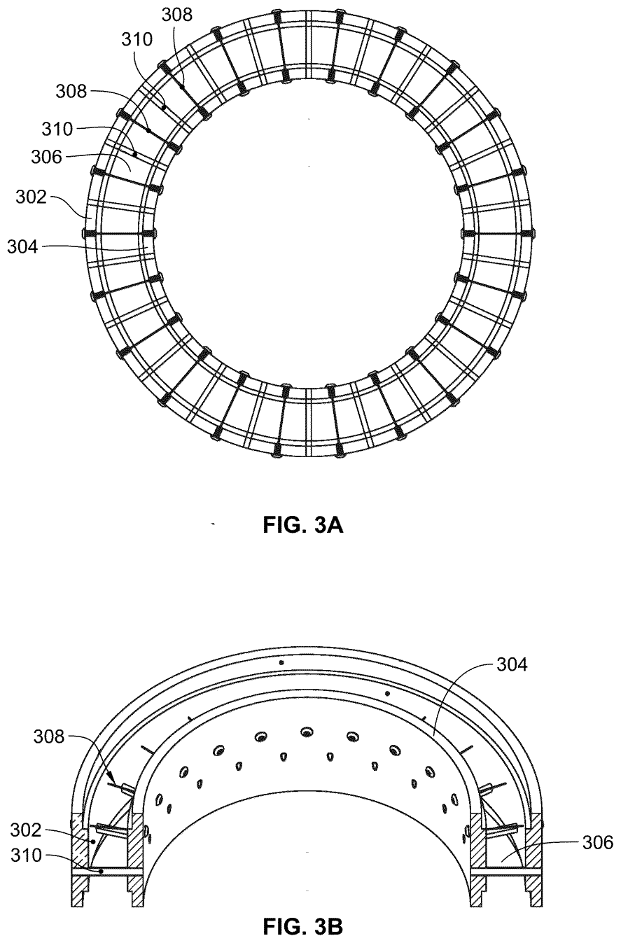

FIG. 3A shows a top view of an ionic thruster assembly 104 of the apparatus, according to an embodiment of the present disclosure. FIG. 3B is a cut-sectional view of the ionic thruster assembly 104 of the apparatus, according to an embodiment of the present disclosure.

Referring to FIG. 3A, the ionic thruster assembly 104 is an annular-shaped assembly comprising an outer cylindrical wall 302 and an inner cylindrical wall 304. The outer cylindrical wall 302 and the inner cylindrical wall 304 are coaxial with different diameters that define an annular space 306 between the outer cylindrical wall 302 and the inner cylindrical wall 304, as shown in FIG. 3B. The ionic thruster assembly 104 may further include a plurality of electrodes riveted between the outer cylindrical wall 302 and the inner cylindrical wall 304. The plurality of electrodes is riveted perpendicular to an axis of the cylindrical walls (302 & 304). Further, the plurality of electrodes is configured across the inner cylindrical wall 304 throughout the annular space 306. The plurality of electrodes is configured to generate a non-thermal plasma within the annular space 306 that works a plasma generation region of the ionic thruster assembly 104.

The plurality of electrodes includes one or more charged electrodes 308 and one or more ground electrodes 310. The charged electrodes 308 and the ground electrodes 310 may be connected to a power source and a large potential difference is applied. Consequently, a partial breakdown of air present within the plasma generation region 306 may be achieved which induces high reactive plasma species including reactive oxygen species (ROS) and reactive nitrogen species (RNS). Such highly reactive plasma species upon contact eliminate micro-organisms present within the air. Further, these plasma species accumulate an electric charge on the particles such as pollutant particles in the airflow.

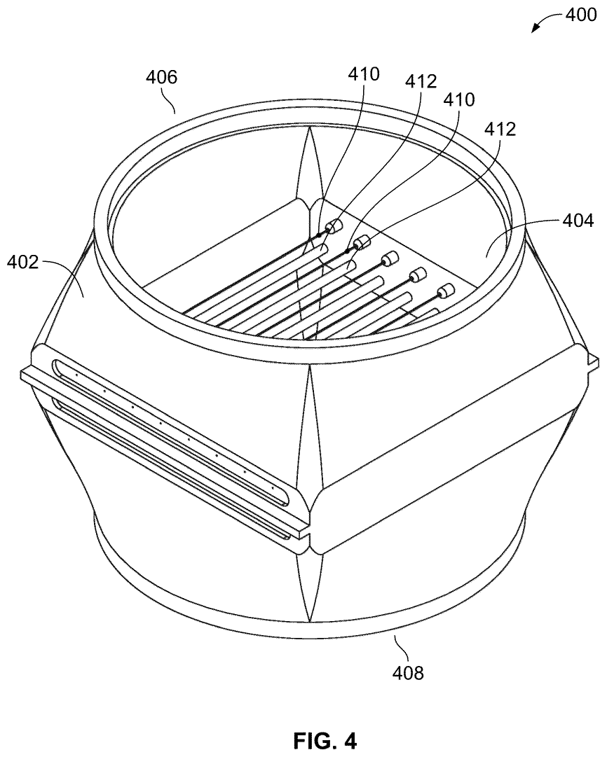

FIG. 4 is a perspective view of the ionic thruster assembly 400 of the apparatus 100, according to another embodiment of the present disclosure.

The ionic thruster assembly 400 may be formed in any geometric shape including, but not limited to, square, circular, rectangular, or octagonal. Referring to FIG. 4, the ionic thruster assembly 400 comprises an octagonal duct 402 with an internal cavity 404. The octagonal duct 402 further comprises an opening 406 at a top end and an opening 408 at a bottom end. The octagonal duct 402 further comprises a plurality of electrodes including one or more charged electrodes 410 and one or more ground electrodes 412 configured within the internal cavity 404 of the octagonal duct 402.

The ionic thruster assembly 400 may be mounted on the swirl generation unit 104. The ionic thruster assembly 400 may be mounted such that the opening 408 at the bottom end of the octagonal duct 402 may be configured over the swirl generation unit 104 aligning the internal cavity 404 of the hexagonal duct 402 with an annular cavity of the swirl generation unit 104. Further, the opening 406 at the top end of the octagonal duct 402 is configured to inlet the airflow within the inner cavity 404. Interchangeably, swirl generation unit 104 may be mounted on the ionic thruster assembly 400,

The plurality of electrodes configured within the inner cavity 404 are connected to a power source (not shown) and applied with a high potential difference resulting in the generation of a non-thermal plasma within the inner cavity 404. The generated non-thermal plasma within the inner cavity 404 disinfects the inlet airflow and accumulates a charge on the particles in the airflow.

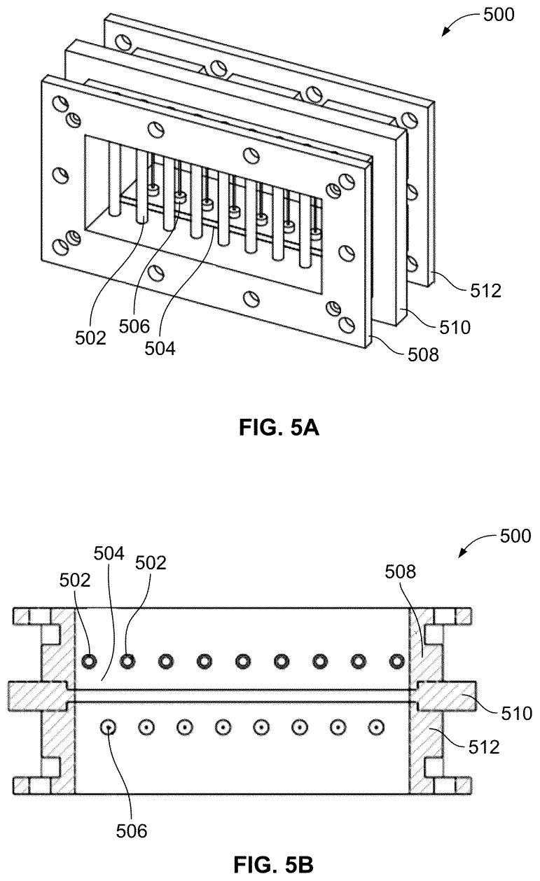

Similarly, FIG. 5A is a perspective view of an electrode configuration 500 for a rectangular-shaped ionic thruster assembly of the ionic thruster assembly, according to another embodiment of the present disclosure. FIG. 5B is a top view of the electrode configuration 500 as shown in FIG. 5A, according to another embodiment of the present disclosure.

Referring to FIG. 5A and FIG. 5B, the electrode configuration 500 may include a rectangular ground electrode cassette 508 comprising a plurality of ground electrodes 502, a rectangular energized electrode cassette 512 comprising a plurality of energized electrodes 506, and an electrode spacer 510 between the ground electrode cassette 508 and the energized cassette 512. The electrode spacer 510 between the ground electrode cassette 508 and the energized cassette 512 creates a plasma generation region 504 between the plurality of ground electrodes 502 and the plurality of energized electrodes 506. Further, the plurality of ground electrodes 502 and the plurality of energized electrodes 506 are configured parallel to each other with the plasma generation region 504 disposed in between. When the plurality of energized electrode 506 and the plurality of ground electrodes 502 are connected to the power supply (not shown) and applied with a large potential difference, non-thermal plasma is generated within the plasma generation region 504 that is used to disinfect the airflow and charge the particles present within the airflow.

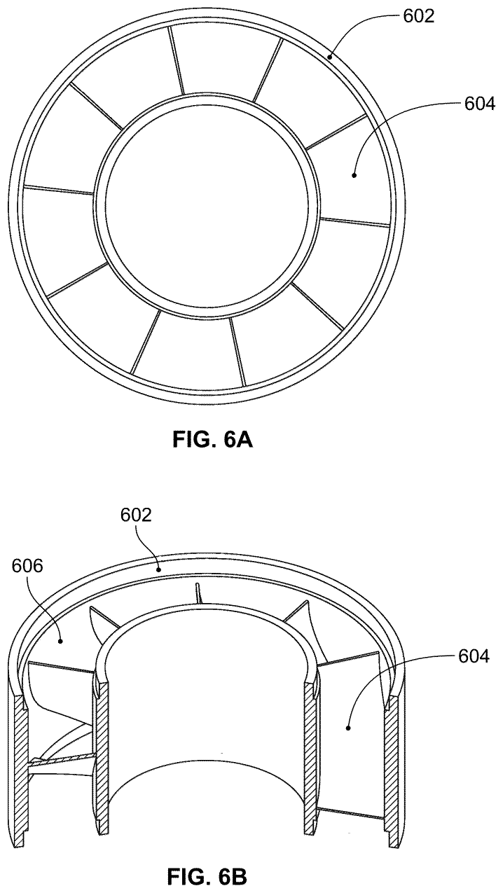

FIG. 6A is a top view of a swirl generation unit 106 of the apparatus 100, according to an embodiment of the present disclosure. FIG. 6B is a cross-sectional view of the swirl generation unit 106 of FIG. 6A, according to an embodiment of the present disclosure.

Referring to FIG. 6A, the swirl generation unit 106 is an annular-shaped device comprising an annular-shaped main body 602 with an annular space 606 (shown in FIG. 6B) and a plurality of static vanes 604 formed equidistantly within the annular space 606 covering the entire annular space 606.

The size of the annular space 606 in a width direction at a bottom end of the annular-shaped main body 602 may be larger than the size of the annular cavity 122 in a width direction at the first end 128 of the electrode section 102. Therefore, the bottom end of the annular-shaped main body 602 sets over the first end 128 of the electrode section 102 when the swirl generation unit 106 is mounted over the electrode section 102. Further, the annular space 606 of the swirl generation unit 106 aligns with the annular cavity of the electrode section 102 such that the airflow from the ionic thruster assembly 104 may flow into the annular cavity 122 of the electrode unit 102 through the annular space 606 of the swirl generation unit 104.

Further, the vanes 604 are provided in a curved shape with a slope formed from a top end to a bottom end of the vanes 604. Further, the slope turns towards an outwards direction from the top end to the bottom end to generate a swirl motion of the airflow within the annular cavity 122 and to direct the airflow towards the outer electrode 118.

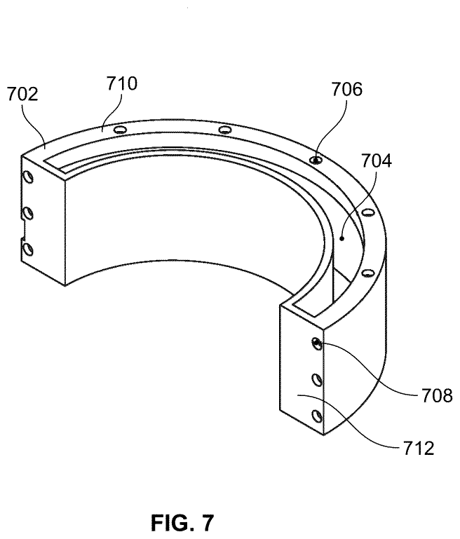

FIG. 7 is a perspective view of a semi-circular half of a particle collector 110, according to an embodiment of the present disclosure.

Referring to FIG. 7, the particle collector 110 is made of two annular-shaped semi-circular halves detachably joined together. Each of the two semi-circular halves includes an annular-shaped body 702 with an opening at a top end and closed from a bottom end defining an annular-shaped enclosed cavity 704 to collect and hold the dust particles. The particle collector 110 may be connected at the second end 130 of the electrode section 102. Further, the particle collector 110 is configured at the second end 120 to cover the bottom opening of the annular cavity 122. Each semi-circular half of the particle collector 110 may include a plurality of magnetic coupling (706 and 708) at a connecting surface (710 and 712).

The semi-circular halves of the particle collector 110 detachably connect with each other from the connecting surface 712 through the plurality of magnetic couplings 708. Further the semi-circular halves of the particle collector 110 detachably connect to the electrode section 102 from the connecting surface 710 through the plurality of magnetic couplings 706. The particle collector 110 collects the particles separated from the airflow and attracted towards an inner surface of the second electrode 118.

The particle collector 110 is detachably connected to enable the user to detach, clean, and re-attach the particle collector to the apparatus 100.

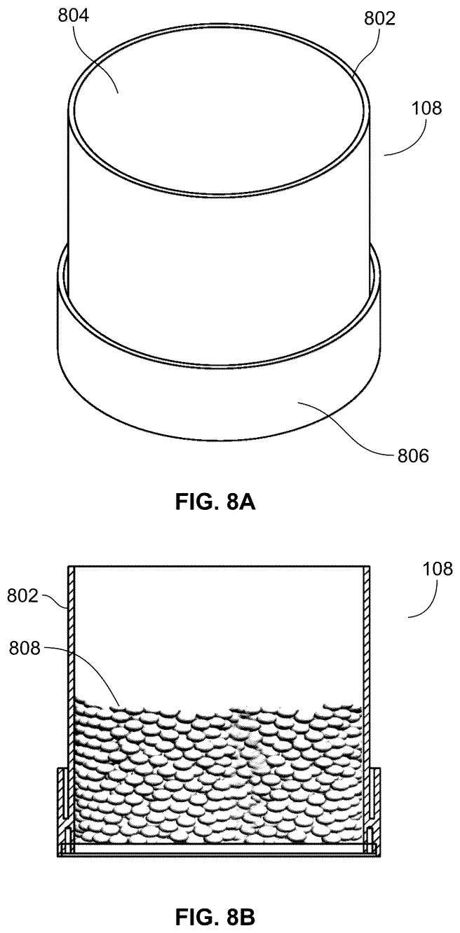

FIG. 8A is a perspective view of a catalytic bed and/or absorbent bed 108 of the apparatus, according to an embodiment of the present disclosure. FIG. 8B is a cross-sectional view of the catalytic bed and/or absorbent bed 108 as shown in FIG. 8A, according to an embodiment of the present disclosure.

Referring to FIG. 8A and FIG. 8B, the catalytic bed and/or absorbent bed 108 is configured to absorb the eliminate ozone from the airflow exhausting out of the apparatus 100. The catalytic bed and/or absorbent bed 108 is configured at the outlet of the inner electrode 120 and includes a cylindrical body 802 with openings at both ends. Further, the catalytic bed and/or absorbent bed 108 include a closure member 806 enclosing an opening at one end to define a hollow cavity 804 within the cylindrical body 802.

Further, the catalytic bed and/or absorbent bed 108 includes a catalytic/adsorbent bed may include an absorbent, an ozone decomposition catalyst, or a combination 808 to absorb the ozone from the exhaust airflow, as shown in FIG. 8B. According to an embodiment, a bottom surface of the closure member may be made of an air-permeable material to enable the airflow to exhaust out of the catalytic bed and/or absorbent bed 108.

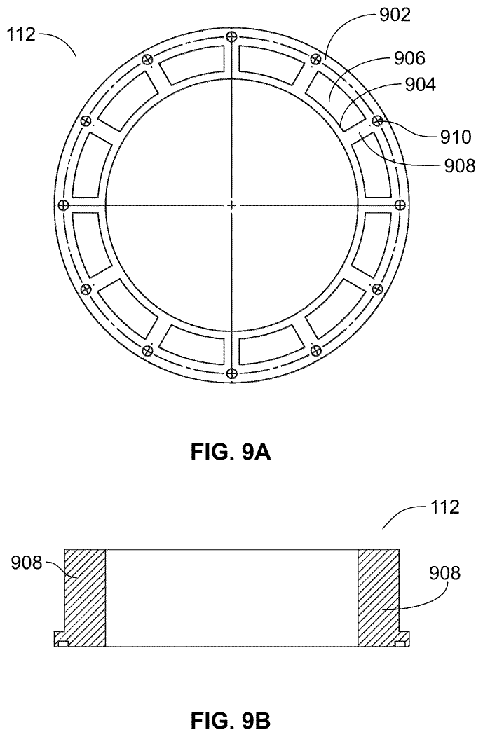

FIG. 9A is a top view of a collector adaptor 112 of the apparatus 100, according to an embodiment of the present disclosure. FIG. 9B is a side cross-sectional view of the collector adaptor 112 of FIG. 9A, according to an embodiment of the present disclosure.

Referring to FIG. 9A and FIG. 9B, the collector adaptor 112 further comprises a cylindrical outer wall 902 and a coaxial inner wall 904 defining an annular space 906 between the outer wall 902 and the inner wall 904. Further, the collector adaptor 112 includes a plurality of vertical members 908 equidistantly connected between the outer wall 902 and the inner wall 904. The plurality of vertical members 908 are connected perpendicular to the axis of the cylindrical walls (902 and 904) and spread across the periphery of the annular space 906. Further, each of the two vertical members 908 is provided with a hollow space in between for the charged particles from the annular cavity 122 to enter into the particle collector 110 via the collector adaptor 112. Further, the collector adaptor 112 may include a plurality of coupling members 910 to connect the collector adaptor 112 to the bottom end 130 of the electrode section 102, within the annular cavity 122. The coupling member 910 may be a magnetic coupling.

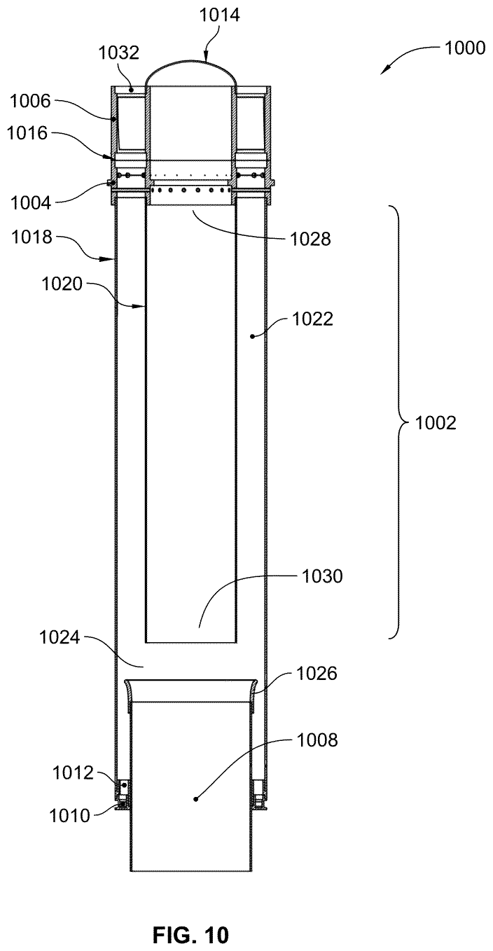

FIG. 10 is a cut-sectional view of an apparatus 1000 for air disinfection and filtration, according to another embodiment of the present disclosure.

Referring to FIG. 10, the apparatus 1000 includes an electrode section 1002 and an ionic thruster assembly 1004 connected in a fluid communication with the electrode section 1002. The ionic thruster assembly 1004 is connected at a first end 1028 of the electrode section 1002. Further, the apparatus 1000 includes a swirl generation unit 1006 connected on the ionic thruster assembly 1004.

The electrode section 1002 further includes an outer electrode 1018 and an inner electrode 1020. The outer electrode 1018 and the inner electrode 1020 are elongated co-axial electrodes. Further, the outer electrode 1018 and the inner electrode 1020 may be cylindrical or conical electrodes with a different diameter, thus defining an annular cavity 1022 between the electrodes.

Further, the ionic thruster assembly 1004 and the swirl generation unit 1006 are annular in shape such that the annular cavities of the ionic thruster assembly 1004 and the swirl generation unit 1006 align with the annular cavity 1022 between the electrodes. Therefore, the air entering from an inlet 1032 of the swirl generation unit 1006 passes through the ionic thruster assembly 1004 into the annular cavity 1022. The ionic thruster assembly disinfects the entered air using a non-thermal plasma and charges the particles present within the air.

The disinfected air with the charged particles passes through the annular cavity 1022 towards a second end 1030 of the electrode section 1002 opposite to the first end 1028.

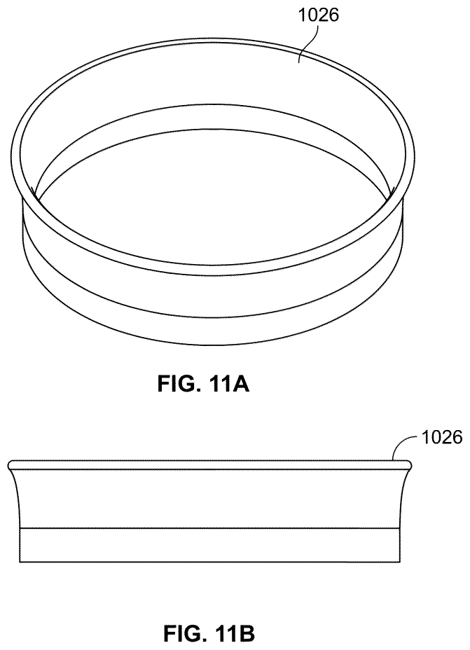

According to the present embodiment, a length of the outer electrode 1018 is higher than a length of the co-axial inner electrode 1020. Therefore, the outer electrode 1028 includes a cylindrical cavity 1024 after the overlapping portion of the electrodes that defines the electrode section 1002. The apparatus 1000 further comprises a catalytic/adsorbent bed 1008 connected at an outlet of the outer electrode 1018. The catalytic/adsorbent bed 1008 is configured within the cylindrical cavity 1024 at a specific distance from a second end 1030 of the electrode section 1002. Further, the catalytic/adsorbent bed 1008 is connected through a connecting member 1026. The connecting member 1026 may be a connecting ring having a cured wall with a different diameter at a top end and a bottom end of the connecting ring as shown in FIG. 11A and FIG. 11B.

1. An apparatus for air disinfection and filtration, comprising:
an electrode section comprising coaxial electrodes with an annular cavity between the coaxial electrodes;
an ionic thruster assembly in fluid communication with the annular cavity of the electrode section at a first end of the electrode section, wherein
the ionic thruster assembly includes a top inlet and is configured to:
receive an airflow through the top inlet;
charge particles present within the airflow entering through the top inlet; and
transmit the airflow with the charged particles into the annular cavity of the electrode section; and
the electrode section is configured to attract the charged particles towards an outer electrode of the coaxial electrodes.

2. The apparatus of claim 1, wherein
the coaxial electrodes include the outer electrode and an inner electrode,
the outer electrode and the inner electrode are hollow, and
a diameter of the outer electrode is larger than a diameter of the inner electrode to define the annular cavity inbetween.

3. The apparatus of claim 2, wherein
the coaxial electrodes are any of a cylindrical electrode or a conical electrode.

4. The apparatus of claim 1, wherein
the ionic thruster assembly further comprises a plurality of electrodes that includes at least one charged electrode and at least one ground electrode.

5. The apparatus of claim 1, wherein
the ionic thruster assembly further comprises:
an outer cylindrical wall;
a coaxial inner cylindrical wall defining an annular space between the outer cylindrical wall and the inner cylindrical wall; and
a plurality of electrodes between the outer cylindrical wall and the inner cylindrical wall, wherein
the plurality of electrodes are configured throughout a periphery of the inner cylindrical wall within the annular space, and
the plurality of electrodes includes at least one charged electrode and at least one ground electrode.

6. The apparatus of claim 5, wherein
in a state in which a potential difference is applied between the at least one charged electrode and the at least one ground electrode, the ionic thruster assembly is further configured to generate a plasma within the annular space between the inner cylindrical wall and the outer cylindrical wall.

7. The apparatus of claim 1, wherein
the ionic thruster assembly further comprises:
a duct having an internal cavity, an opening at a top end, and an opening at a bottom end opposite to the top end; and
a plurality of electrodes within the internal cavity, wherein
the duct is at least in a shape of square, circular, rectangular, and
the plurality of electrodes includes at least one charged electrode and at least one ground electrode.

8. The apparatus of claim 1, further comprises:
a swirl generation unit between the electrode section and the ionic thruster assembly; or
the ionic thruster assembly between the swirl generation unit and the electrode section.

9. The apparatus of claim 8, wherein
the swirl generation unit further comprises:
an annular-shaped main body; and
a plurality of static vanes equidistantly configured covering an annular space of the annular-shaped main body.

10. The apparatus of claim 9, wherein
the swirl generation unit is configured to:
generate a swirl motion of the airflow with the charged particles within the annular cavity between the coaxial electrodes;
direct the airflow toward the outer electrode of the coaxial electrodes.

11. The apparatus of claim 2, wherein
the inner electrode and the outer electrode are applied with a large potential difference of opposite polarities to attract the charged particles present within the airflow to an inner surface of the outer electrode.

12. The apparatus of claim 2, wherein
the inner electrode further comprises:
one or more apertures within a periphery; and
an outlet at an opposite end to the first end of the electrode section, wherein
the one or more apertures are near the outlet of the inner electrode.

13. The apparatus of claim 12, wherein
the one or more apertures are configured for the airflow to transit from the annular cavity between the coaxial electrodes to an inner cavity of the inner electrode, and
the outlet is configured to exhaust the airflow out of the apparatus.

14. The apparatus of claim 2, wherein
a length of the outer electrode is higher than a length of the inner electrode.

15. The apparatus of claim 13, further comprises:
at least one catalytic bed or an absorbent bed at the outlet, wherein
the catalytic bed or the adsorbent bed is configured to eliminate ozone from the exhausted airflow.

16. The apparatus of claim 14, further comprises:
at least one catalytic bed or an absorbent bed at an outlet of the outer electrode, wherein
the catalytic bed or the absorbent bed is configured at a distance from the inner electrode to define a cylindrical cavity between the catalytic bed or the absorbent bed and the inner electrode.

17. The apparatus of claim 1, further comprises
a particle collector at an end opposite to the first end of the electrode section, wherein
the particle collector is configured to collect the charged particles attracted at the outer electrode.

18. The apparatus of claim 17, wherein
the particle collector is annular in shape and detachably connected to the annular cavity of the electrode section, and
in a state in which the particle collector is mounted on the electrode section, the particle collector covers the annular cavity at the end opposite to the first end of the electrode section.

19. The apparatus of claim 17, wherein
the particle collector is detachably connected to the electrode section through a magnetic coupling.

20. The apparatus of claim 19, wherein
the particle collector comprises two mirrored semi-circular halves detachably connected to form the annular-shaped particle collector, and
the two mirrored semi-circular halves are connected through a magnetic coupling.

21. The apparatus of claim 17, further comprises:
a collector adaptor between the particle collector and the electrode section.

22. The apparatus of claim 21, wherein
the collector adaptor is configured to break a swirl motion within the annular cavity of the electrode section.

23. The apparatus of claim 2, further comprises:
a dome-shaped closure member enclosing a top opening of the inner electrode, wherein
the dome-shaped closure member is configured to guide airflow into the annular cavity between the coaxial electrodes.
